(19) United States
(12) Patent Application Publication
(10) Pub. No.: US 2018/0111080 A1
(43) Pub. Date: Apr. 26, 2018

(54) AIR PURIFYING APPARATUS

(71) Applicant: Seong Hun Lee, Changwon-si, Gyeongsangnam-do (KR)

(72) Inventor: Seong Hun Lee, Changwon-si, Gyeongsangnam-do (KR)

(21) Appl. No.: 14/893,490

(22) PCT Filed: Jul. 15, 2015
(86) PCT No.: PCT/KR2015/007347

§ 371 Date: Dec. 13, 2017

(30) Foreign Application Priority Data
Apr. 8, 2015 (KR) 10-2015-0049928

(51) Int. Cl. B01D 47/06; B01D 50/00; B01D 53/86; C02F 11/12; A61L 9/20; A61L 9/00

(52) U.S. Cl. CPC B01D 47/06; B01D 50/006; B01D 53/86; C02F 11/127; C02F 2103/18; A61L 9/00; B01D 2255/802; B01D 2255/20707; A61L 9/205

(57) ABSTRACT

The present invention provides an air purifying apparatus that can continuously discharge sludge separated from suctioned contaminated air from a dust collecting apparatus to the outside, thereby improving convenience of use and air purifying performance, and can mix contaminated air with water, eliminate the sludge and contaminants in the air, and allow the eliminated sludge and contaminants to pass through a photocatalytic purifying unit and purify air using a photocatalytic reaction, thereby improving air purifying performance.

### TECHNICAL FIELD

The present invention relates to an air purifying apparatus.

### BACKGROUND ART

In general, an air purifying apparatus is an apparatus for eliminating contaminants such as floating particles in the air, germs, and toxic gases to maintain a cleanliness of an environmental space, and may purify air using various methods such as oxidation, reduction, decomposition, adsorption, filtering, electrical collection, and washing.

In particular, the air purifying apparatus may include a dust collecting apparatus, and may be variously used in industrial fields, cattle sheds, workings, homes, offices, and restaurants.

As an example of a dust collecting apparatus, Korean Patent No. 10-1296243 discloses an air purifying wet dust collecting apparatus in which air introduced from the outside is continuously cooled by circulating coolants and sludge and foreign substances are eliminated so that contaminated air can be cooled and the sludge in the air can be efficiently eliminated.

However, according to the related art, sludge is continuously collected in the interior of the dust collecting apparatus when the dust collecting apparatus is used for a long time and a separate operation should be performed to processing the sludge, which is inconvenient.

Further, as another example of an air purifying apparatus, Korean Utility Model No. 20-02546111 discloses a purifying apparatus for water and air in which a photocatalytic plate is formed spirally and a light emitting lamp passes through the center of the photocatalytic plate such that air and water flows along a spiral passage formed by the photocatalytic plate to contact the photocatalytic plate so that the purifying capability of the contaminated water and air can be improved by widening the contact area of the contaminants in the water and air.

However, according to the related art, only air purification due to a photocatalytic reaction is simply allowed and direct separation and elimination of contaminants is impossible, and in particular, it is impossible to add or combine a light emitting lamp according to a demand of the user, making it impossible to implement various requirements.

### SUMMARY OF INVENTION

### Technical Problem

The present invention provides an air purifying apparatus that can continuously discharge sludge separated from suctioned contaminated air from a dust collecting apparatus to the outside, thereby improving convenience of use and air purifying performance.

The present invention also provides an air purifying apparatus that can mix contaminated air with water, eliminate the sludge and contaminants in the air, and allow the eliminated sludge and contaminants to pass through a photocatalytic purifying unit and purify air using a photocatalytic reaction, thereby improving air purifying performance.

### Solution to Problem

In accordance with an aspect of the present invention, there is provided an air purifying apparatus comprising: a case that forms an external appearance of the air purifying apparatus and has a discharge hole through which purified air is discharged; a main blower that is communicated with the interior of the case such that contaminated air flows into the case; a water supply pipe that is provided at an outlet of the main blower to supply water mixed with the contaminated air introduced into the case; a sludge separation unit that is provided in an interior space of the case to form a passage in which the contaminated air mixed with the water introduced into the case makes continuous collisions and to separate the water in the contaminated air and the sludge; a barrier plate that partitions the interior space of the case into an upper air introduction unit and a lower sludge processing unit below the sludge separation unit and has a passage through which the water and the sludge separated from the contaminated air passes; and a sludge discharge unit that is provided in the sludge processing unit to continuously separate the water and the sludge passing through the base plate by rotation thereof and to discharge the separated sludge to the outside of the case.

A filter assembly that purifies air discharged through the discharge hole that is an opening in the case above the sludge separation unit is provided between the discharge hole and the sludge separation unit.

The sludge separation unit includes a plurality of plate-shaped structures that is arranged in a direction perpendicular to a flow direction of air introduced from an outlet of the main blower.

The sludge separation unit is formed along a circumference of the case, and a plurality of holes, through which water and sludge are dropped, are formed in an inner area of the sludge separation unit in the barrier plate.

A water tray that guides the water and the sludge moved downwards to one side of the sludge discharge unit is provided on a lower surface of the barrier plate.

The water supply pipe is connected to a water supply pump that circulates the water collected in the sludge processing unit.

The sludge discharge unit comprises: a drum that is rotated in the sludge processing unit and has a plurality of holes on an outer surface thereof; an air nozzle that is provided inside the drum to eject air supplied by a sludge blower provided in the case and to spray the sludge attached to the outer surface of the drum; a sludge guide that is provided outside the drum facing the air nozzle to guide the sprayed sludge; a screw feeding unit that is provided below the sludge guide to horizontally move the sludge collected by the sludge guide in a screwed manner; and a sludge box that is connected to the screw feeding unit to store the fed sludge.

The air nozzle, the sludge guide, and the screw feeding unit all extend in an axial direction of the drum.

The screw feeding unit comprises: a screw member that is directly connected to a screw motor provided in the case and has a spiral shape to feed the sludge while being rotated; and a screw case that accommodates at least a portion of the screw member below the screw member.

The sludge discharge unit comprises: a drum that is rotated in the sludge processing unit and has a plurality of holes on an outer surface thereof; a sludge box that is provided outside the case to accommodate the sludge fed in the sludge processing unit; a suction nozzle that is communicated with the sludge box and extends to the outside of the drum to suction the sludge attached to the outer surface of the drum; and a suction blower that is communicated with the sludge box to provide a suction force to the suction nozzle.

The opened end of the suction nozzle extends along a lengthwise direction of the drum.

The suction blower supplies the discharged air to the main blower or a passage communicated with the main blower.

The sludge discharge unit comprises: a screen that at least partially partitions the interior of the sludge processing unit below the barrier plate and has a plurality of holes to separate the sludge and the water; a screen motor that is connected to the screen and allows the screen to be rotated while having a rotary shaft crossing the dropping direction of the water; a sludge box that is provided outside the case to accommodate the sludge fed in the sludge processing unit; a suction nozzle that is communicated with the sludge box and extends to the upper surface of the screen to suction the sludge attached to the upper surface of the screen; and a suction blower that is communicated with the sludge box to provide a suction force to the suction member.

A water guide that forms an inclined surface that extends towards the outside as it goes downwards is further formed between the barrier plate and the screen.

A screen bracket that supports an end of the screen such that the screen is rotatably mounted is further formed inside the case.

The screen comprises: a plate that has a net shape to filter sludge and the center of which is opened; an outer frame that is formed along an outer periphery of the plate to accommodate an end of the screen bracket; and an inner frame that is formed along the interior of the plate to accommodate an end of the water guide.

An inlet through which the water and the sludge are discharged downwards is formed in the barrier plate and the inlet is located at a location spaced apart from the suction member.

A blocking plate that blocks the suction member from the inlet is provided between the barrier plate and the screen.

A photocatalytic module for purifying the discharged air is provided at an upper portion of the air flow unit, and the photocatalytic module includes a plurality of photocatalytic plates coated with titanium dioxide and at least one ultraviolet lamp that passes through the plurality of photocatalytic plates.

A negative ion generating unit for neutralizing the discharged air is further provided at an upper portion of the air flow unit.

The air purifying apparatus further comprises: a photocatalytic purifying unit for purifying the air from which water and sludge are separated on an outer side of the case, and the photocatalytic purifying unit comprises a purifying unit in which a plurality of photocatalytic modules each comprising a plurality of photocatalytic plates coated with titanium dioxide and at least one ultraviolet lamps passing through the plurality of photocatalytic plates are vertically arranged in parallel to each other.

A plurality of purifying unit are arranged in parallel in the photocatalytic purifying unit, and a base that is communicated with the plurality of purifying units to form flow passages of the air between the plurality of purifying units is further provided.

A door that is provided to be opened and closed such that an outer end of the ultraviolet lamp is exposed is provided in the purifying unit.

The photocatalytic module is arranged such that a surface of the photocatalytic plate, into which the ultraviolet lamp is inserted, is exposed when the door is opened.

The plurality of photocatalytic modules is vertically stacked such that ends of the vertically adjacent photocatalytic plates contact each other to connect the flow passages of the air.

A central portion of the photocatalytic plates is bent, and opposite sides of the photocatalytic plates are inclined.

A plurality of air holes are punched over the front surface of the photocatalytic plate, and the photocatalytic plate is bent along a central portion thereof and opposite side surfaces thereof are inclined with respect to the central portion thereof.

One or more lamp mounting recesses that are recessed such that a hole into which the ultraviolet lamp is inserted is formed when the plurality of photocatalytic modules are stacked are formed at adjacent ends of the plurality of photocatalytic modules.

The air purifying unit comprises: a case that forms an external appearance of the air purifying apparatus and has a discharge hole through which purified air is discharged; an introduction duct that supplies contaminated air into the case; an internal motor that is provided in the interior of the case and a rotary shaft of which extends vertically; a suction fan that is connected to an upper end of the rotary shaft to suction the contaminated air; a water supply pipe that supplies water to the contaminated air flowing below the suction fan; an impeller that is connected to a lower end of the rotary shaft to collide with the contaminated air mixed with the water flowing downwards; a barrier plate that partitions the interior space of the case into an upper air introduction unit and a lower sludge processing unit below the impeller and has a passage through which the water and the sludge separated from the contaminated air passes; and a sludge discharge unit that is provided in the sludge processing unit and which the mixed water and sludge passing through the barrier plate continuously contact a porous member such that the water passes and the sludge is separated while being attached, the separated sludge being discharged to the outside of the case.

The diameter of the impeller is larger than the diameter of the suction fan.

The water supply pipe is arranged along the circumference of the internal motor between the suction fan and the impeller such that the water is ejected to the impeller.

The internal motor is positioned in the motor mount mounted to the barrier plate.

A motor cover that surrounds the motor to form a space for accommodating the motor is provided in the interior of the case, and the introduction duct is communicated with the interior of the motor cover.

### Advantageous Effects of Invention

The air purifying apparatus according to the embodiments of the present invention may expect the following effects.

The air purifying apparatus according to the embodiments of the present invention includes a sludge discharge unit capable of automatically discharging sludge at a lower portion of the dust collecting apparatus to automatically discharge the sludge generated while the dust collecting apparatus is driven.

Accordingly, because a separation manipulation or operation for discharging sludge may not be performed, convenience of use can be improved.

Further, because the sludge in the interior of the dust collecting apparatus is continuously discharged to the outside, an effect of preventing lowering of the performance of the dust collecting apparatus or a secondary contamination generated as the sludge is excessively collected in the interior of the dust collecting apparatus can be expected.

In addition, the contaminated air mixed with water continuously collides with a wall surface of the sludge separation unit provided in the interior of the case so that the separation performance of the sludge can be improved, and accordingly, air purifying performance can be improved.

Furthermore, because the air discharged from the dust collecting apparatus passes through the photocatalytic purifying unit, the sterilizing performance of the discharged air can be improved, and accordingly, the overall air purifying performance can be improved.

In particular, because the photocatalytic purifying apparatuses are stacked in parallel, the flowing air passes through a passage formed by the photocatalytic plates, and because the shape of the photocatalytic plates is bent, the adjacent photocatalytic plates can be guided and communicated with each other in zigzags, so that a contact area of the air can be increased and sterilizing performance can be improved.

Furthermore, because the ultraviolet lamp can be naturally exchanged by opening the door of the photocatalytic purifying unit, the ultraviolet lamp can be selected according to the required sterilizing performance, so that sterilization can be efficiently performed.

### BRIEF DESCRIPTION OF DRAWINGS

FIG. 1 is a front view of an air purifying apparatus according to a first embodiment of the present invention.

FIG. 2 is a left side view of the air purifying apparatus.

FIG. 3 is a right side view of the air purifying apparatus.

FIG. 4 is a rear view of the air purifying apparatus.

FIG. 5 is a side view illustrating a structure of a sludge processing unit according to the first embodiment of the present invention.

FIG. 6 is a front view illustrating the structure of the sludge processing unit.

FIG. 7 is a side view illustrating a structure of a sludge processing unit according to a first embodiment of the present invention.

FIG. 8 is a front view illustrating the structure of the sludge processing unit.

FIG. 9 is a side view illustrating a structure of a sludge processing unit according to a third embodiment of the present invention.

FIG. 10 is a side view of the sludge processing unit.

FIG. 11 is a plan view of the sludge processing unit.

FIG. 12 is a front view illustrating a structure of an air flow unit according to a fourth embodiment of the present invention;

FIG. 13 is a side view of the air flow unit.

FIG. 14 is a front view illustrating a structure of an air flow unit according to a fifth embodiment of the present invention.

FIG. 15 is a front view of an air purifying apparatus according to a sixth embodiment of the present invention.

FIG. 16 is an exploded perspective view of a photocatalytic module according to the sixth embodiment of the present invention.

FIG. 17 is a plan view of a photocatalytic plate that is a main part of the photocatalytic module;

FIG. 18 is a partially sectional view of the photocatalytic module.

FIG. 19 is a view illustrating an air flow in the photocatalytic module.

### DESCRIPTION OF EMBODIMENTS

Hereinafter, an air purifying apparatus according to exemplary embodiments of the present invention will be described in detail with reference to the accompanying drawings.

Although suctioned air is limited to contaminated air in the embodiments of the present invention, it encompasses vapor containing contaminants or contaminated air containing moisture, and will be referred to as contaminated air for convenience' sake in the following.

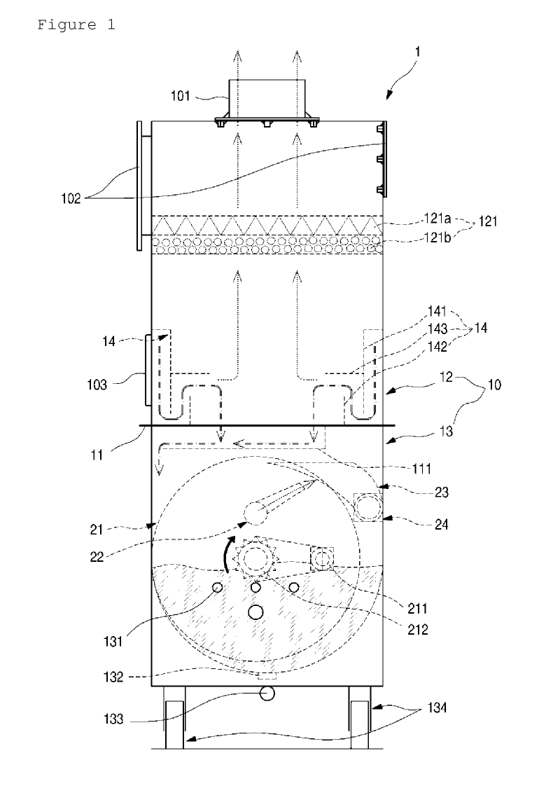

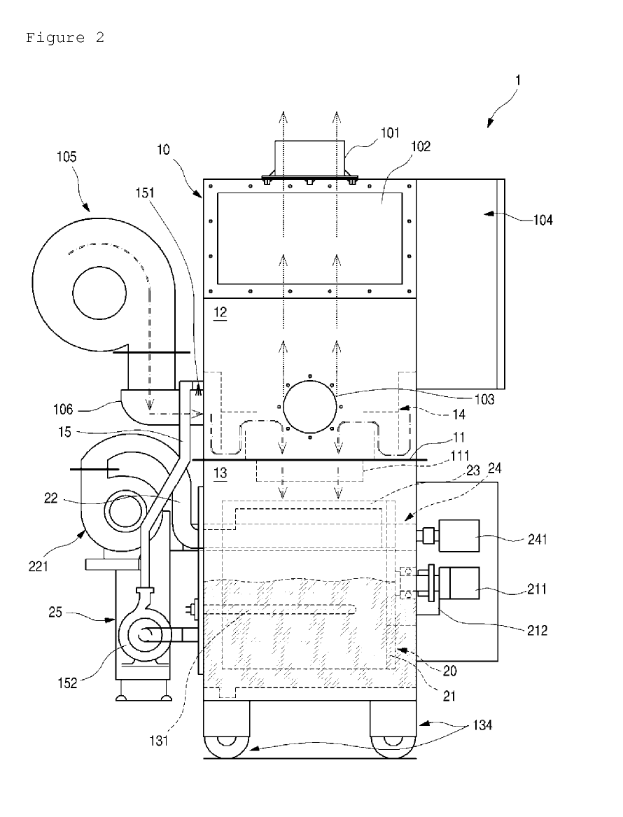

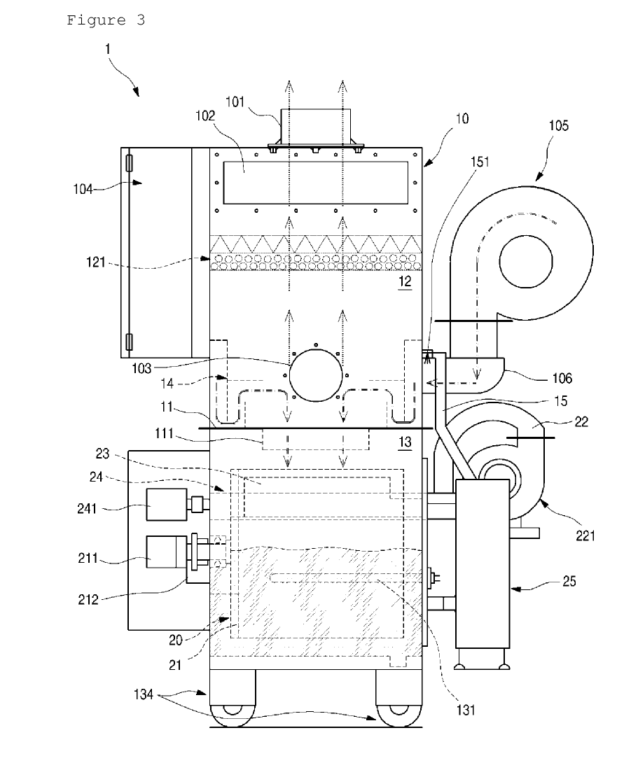

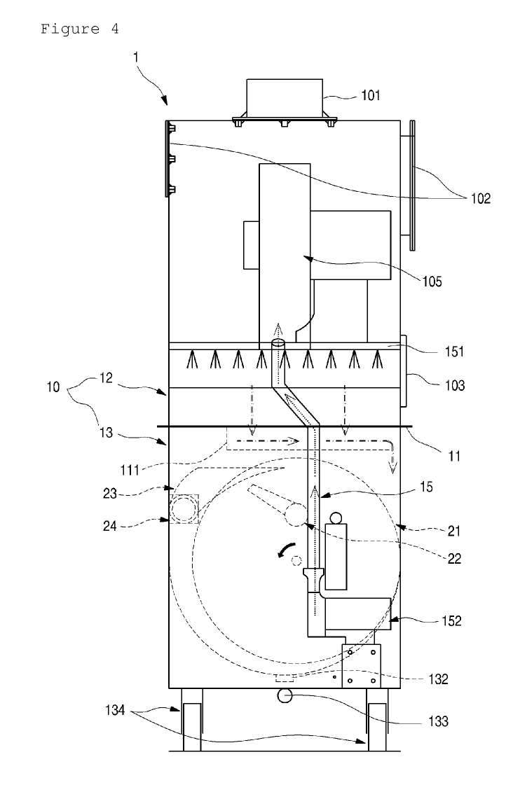

FIG. 1 is a front view of an air purifying apparatus according to a first embodiment of the present invention. FIG. 2 is a left side view of the air purifying apparatus. FIG. 3 is a right side view of the air purifying apparatus. FIG. 4 is a rear view of the air purifying apparatus.

Referring to FIGS. 1 to 4, an external appearance of the air purifying apparatus 1 according to the embodiment of the present invention is formed by a case 10.

The case 10 has a space for flows of air for purification and discharge of sludge in the interior thereof. The case 10 may be variously configured according to the scale of the duct collecting apparatus 1.

The case 10 may include an air flow unit 12 and a sludge processing unit 13 on the upper and lower sides of a barrier plate 11 provided in the interior thereof, respectively.

A discharge hole 101 through which purified air is discharged is formed at an upper end of the case 10. The discharge hole 101 may be configured to be connected to a separate duct or another apparatus for additional purification of air.

An upper monitoring window 102 through which a discharge state of the purified air may be monitored may be provided at an upper end of the case 10, that is, at an upper end of the air flow unit 12. One side monitoring window 102 of the upper monitoring window 102 may be opened and closed, and services such as checking of the interior of the case 10 and replacement of a filter assembly 121 may be enabled by opening and closing the upper monitoring window 102.

One side monitoring window 102 of the upper monitoring window 102 is opened when a photocatalytic purifying unit 60 is connected thereto such that a discharge pipe 18 is connected to the monitoring window 102 to introduce the air discharged from the duct collecting apparatus 1 into the photocatalytic purifying unit 60 through the discharge pipe 18.

A filter assembly 121 may be provided at an upper portion of the case 10. The filter assembly 121 is adapted to eliminate foreign substances and spray water in the air discharged to the outside, and may include a combination of a plurality of filters. For example, the filter assembly 121 may be a combination of a free filter 121a that filters foreign substances, and a functional filter 121b to which a filtering agent having a specific function is added. The filter assembly 121 may be replaceably provided through opening and closing of the upper monitoring window 102.

A lower monitoring window 103 through which a state of air passing through a sludge separating unit 14, which will be described below, may be monitored may be provided at a lower portion of the air flow unit 12. The lower monitoring window 103 may be provided at the same height as that of the sludge separating unit 14, and may be configured to be openable.

Meanwhile, a control box 104 may be provided on the outside of an upper portion of the case 10. The control box 104 is adapted to supply electric power to the dust collecting apparatus 1 and to set and control an operation of the dust collecting apparatus 1, and may be configured to be exposed to the outside such that the user may approach the control box 104.

A main blower 105 may be provided on the outside of an upper portion of the case 10. The main blower 105 may suction contaminated air and compulsorily supply the suctioned air into the case 10, and may include an impeller generally used for a blower and a housing that accommodates the impeller to guide a flow of air.

An inlet of the main blower 105 through which the contaminated air is suctioned is exposed such that exterior air may be suctioned, and may be connected to a separate duct such that air in a contaminated space may be introduced into the interior of the case 10 through the main blower 105.

An outlet of the main blower 105 may be communicated with one side of the air flow unit 12 corresponding to the location of the sludge separation unit 14. To achieve this, a connection duct 106 that connects the outlet of the main blower 105 and the opening of the case 10 may be further provided.

A water supply pipe 15 may be connected to an outlet of the main blower 105, more particularly, to the connection duct 106, and a water supply nozzle 151 that injects water into the connection duct 106 may be provided at an end of the water supply pipe 15.

Accordingly, the contaminated air discharged from the main blower 105 may be introduced into the case 10 while being mixed with the water supplied from the water supply nozzle 151. Then, the water supply nozzle 151 may be located at the outlet of the main blower 105 instead of the connection duct 106, and may be provided at one side where air is introduced into the case 10.

Meanwhile, the water supply pipe 15 is connected to the water supply pump 152, which will be described below, and the water supply pump 152 is communicated with the sludge processing unit 13 to circulate the water in the case 10.

The sludge separation unit 14 may be provided in the case 10 corresponding to the main blower 105. The sludge separation unit 14 may be formed along an inner peripheral surface of the case 10 and the bottom of the air flow unit 12, and the flow direction of the air introduced into the case 10 may be continuously changed a plurality of times in the flow process so that sludge may be separated from the contaminated air mixed with water.

In detail, the sludge separation unit 14 may include a first passage 141 formed along a circumference of an inner surface of the case 10 such that the air suctioned from one side of the case 10 flows downwards, a second passage 142 located on the inner side of the case 10 than the first passage 141 and extending upwards from the barrier plate 11 such that the air passing through the first passage 141 flows upwards again, and a third passage 143 arranged on the upper side of the opened upper surface of the second passage 142 and extending from one side of the first passage 141 to the inner side of the case 10 such that cold air passing through the second passage 142 flows downwards again.

The first passage 141, the second passage 142, and the third passage 143 may be formed of a plate-shaped material, and may be formed along the circumference of the case 10 or the circumference of the barrier plate 11 with respect to the inner center of the case 10.

The contaminated air introduced into the case 10 by the main blower 105 flows along the first passage 141, the second passage 142, and the third passage 143, and finally flows to the center of the case 10.

Then, the contaminated air introduced into the case 10 collides with the first passage 141, the second passage 142, and the third passage 143 while being sprayed after being mixed with water, and in particular, the contaminated air mixed with water strongly collides with the inner surface of the sludge separation unit 14 at a part where the flow direction thereof is changed. Accordingly, those of the sludge in the contaminated air and other contaminants, which have large particles, are separated from air in the process of passing through the sludge separation unit 14, and are dropped and flow towards the barrier plate 11.

In the process of passing through the sludge separation unit 14, the sludge is separated together with water and is dropped downwards, and the air from which sludge and water are eliminated flows from the center of the case 10 upwards and is discharged through the outlet 101 after passing through the filter assembly 121.

Meanwhile, the barrier plate 11 may partition the interior of the case 10 and an area except for the circumference thereof has a mesh shape or an opened shape so that the sludge and water that are dropped while passing through the sludge separation unit 14 may be discharged.

A water tray 111 that guides the water passing through the barrier plate 11 to flow in one direction is provided on the lower surface of the barrier plate 11. One side of the water tray 111 has an opened drawer shape, and an opening of the water tray 111 is opened towards the sludge processing unit 13. The opening of the water tray 111 prevents the water and sludge passing through the barrier plate 11 from being introduced into a screw feeding unit 24, which will be described below.

A sludge discharge unit 20 may be provided in the sludge processing unit 13. The sludge discharge unit 20 may include a drum 21 to which sludge is attached, an air nozzle 22 that separates the sludge attached to the drum 21 by ejecting air to the drum 21, a sludge guide 23 that guides the sprayed sludge, and a screw feeding unit 24 that moves the sludge collected by the sludge guide 23 to a sludge box 25.

Meanwhile, a cylindrical drum 21 is provided in the sludge processing unit 13 below the case 10. The drum 21 is configured to occupy almost all portions of the interior of the sludge processing unit 13, and may be rotated by a drum motor 211 mounted on an outside of the case 10. Then, the drum motor 211 and the drum 21 may be connected by a reducer 212, and the rotational speed of the drum 21 may be adjusted. A plurality of vent holes are formed on an outer peripheral surface of the drum 21, and at least a portion of the outer peripheral surface of the drum 21 may have a mesh or net shape. Accordingly, the water supplied to the drum 21 while being mixed with the sludge may be discharged through the holes formed on the surface of the drum 21, and the sludge may be adsorbed on the outer surface of the drum 21.

A screw motor 241 for driving the screw feeding unit 24 may be provided in the case 10, and the screw motor 241 may be provided separately from the drum motor 211 to be driven independently. Of course, if necessary, the drum 21 and the screw feeding unit 24 may be rotated by one motor.

An ultraviolet lamp 131 may be provided at one side of the case 10. At least one ultraviolet lamp 131 is arranged below the sludge processing unit 13, and may be inserted from an outer side of the case 10 and extend into the case 10. The ultraviolet lamp 131 remains submerged in the water accommodated in the sludge processing unit 13, and sterilizes the water accommodated in the sludge processing unit 13.

The water supply pipe 15 is connected to one side of the case 10, and a water supply pump 152 communicated with a lower portion of the sludge processing unit 13 is provided. The water accommodated in the sludge processing unit 13 may be supplied to an exit of the main blower 105, that is, to the sludge separation unit 14 through the water supply pipe 15 by the water supply pump 152, and may continuously circulate.

The shape of the lower surface of the sludge processing unit 13, that is, the lower surface of the case 10 may be rounded. A water outlet 132 communicated with the lower surface of the sludge processing unit 13 may be formed on the lower surface of the case 10, and the water accommodated at a lower portion of the sludge processing unit 13 may be discharged to the outside through selective opening of the outlet valve 133.

A caster 134 that facilitates movement of the dust collecting apparatus 1 may be further provided on the lower surface of the case 10.

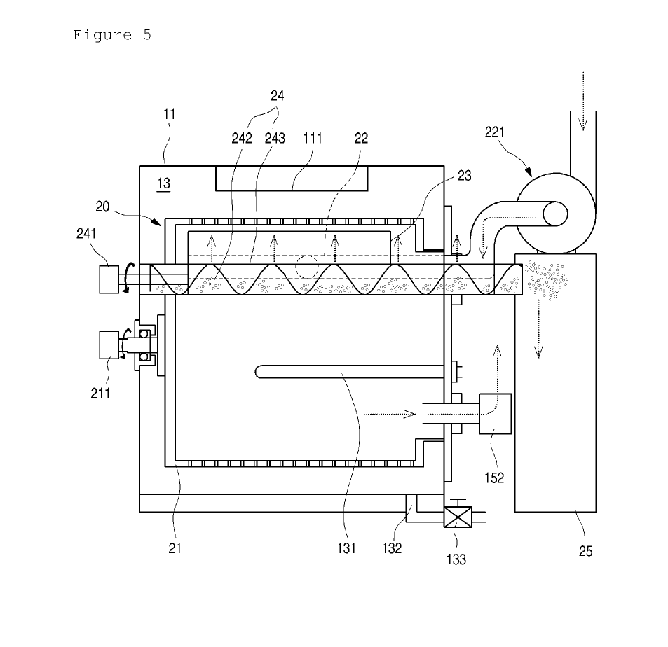

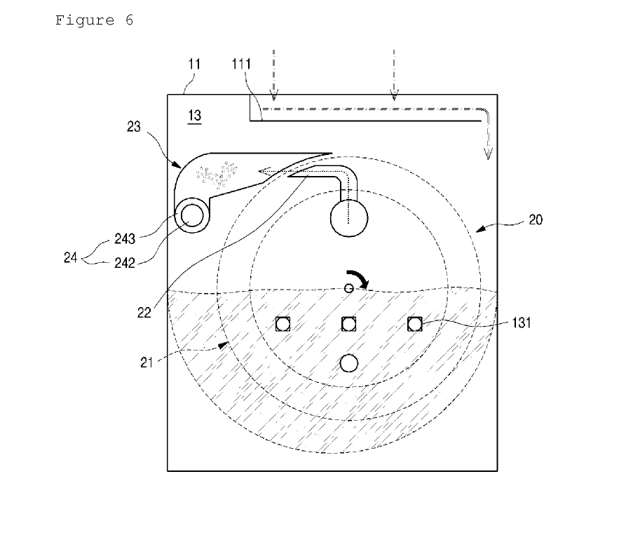

FIG. 5 is a side view illustrating a structure of a sludge processing unit according to the first embodiment of the present invention. FIG. 6 is a front view illustrating the structure of the sludge processing unit.

Referring to the drawings, in more detail, in the structure of the sludge processing unit 13, the air nozzle 22 is provided in the interior of the drum 21 and is connected to a sludge blower 221 provided outside the case 10. The sludge blower 221 may be configured to suction exterior air and blow the air towards the drum 21. The air nozzle 22 is connected to the exit of the sludge blower 221 and an end of the air nozzle 22 from which air is discharged may extend along a lengthwise direction of the drum 21. Then, the extension length of the air nozzle 22 may correspond to the length of the drum 21, and may correspond to the length of the sludge guide 23.

The drum 21 may be arranged to be eccentric from an inside of the sludge processing unit 13 to one side, and the sludge guide 23 and the screw feeding unit 24 may be arranged in a space at an upper portion of an outside of the drum 21 formed by the eccentricity of the drum 21.

The sludge guide 23 may extend from a location adjacent to the outer surface of the drum 21 to the screw feeding unit 24, and may be situated at a location corresponding to an end of the air nozzle 22. The sludge guide 23 may have a length corresponding to an end of the air nozzle 22. Accordingly, the sludge attached to the outer surface of the drum 21 is sprayed by the air ejected by the air nozzle 22 and moves towards the sludge guide 23, and the sludge moved by the sludge guide 23 moves to the screw feeding unit 24.

The screw feeding unit 24 may include a screw motor 241 mounted on the case 10, a screw member 242 connected to the screw motor 241 to be rotated, and a screw case 243 that accommodates the screw member 242.

The screw member 242 and the screw case 243 may be provided in the interior of the case 10, and may have a length corresponding to the sludge guide 23. The screw case 243 and an opposite end of the screw member 242 pass through the case 10 and extend to the sludge box 25 provided outside the case 10. Accordingly, the sludge in the interior of the case 10 fed by rotation of the screw member 242 may be fed to the sludge box 25.

Hereinafter, an operation of the dust collecting apparatus according to the first embodiment of the present invention having the above-mentioned structure will be described.

The contaminated air may be compulsorily introduced into the case 10 by driving the main blower 105. Then, the contaminated air is introduced into the case 10 while being mixed with water by the water supply nozzle 151 provided at an exit of the main blower 105.

The contaminated air mixed with the water introduced into the case 10 flows along the sludge separation unit 14. Then, the contaminated air mixed with water flowing along the passage of the sludge separation unit 14 flows while colliding with a wall surface of the sludge separation unit 14, and accordingly, the sludge or oil vapor contained in the air is separated together with the water.

The air, sludge, and water may be separated while passing through the sludge separation unit 14, and the heavy sludge and water are dropped and the air passes through the sludge separation unit 14 to flow to the upper side of the case 10.

The air flowing upwards is moved while contaminant particles in the air are eliminated, and foreign substances and sprayed water are filtered once again while passing through the filter assembly 121. The air passing through the filter assembly 121 may be finally discharged to the outside through the discharge hole 101.

In the process of passing through the sludge separation unit 14, the separated sludge or water is moved downwards by the weight thereof while being sprayed and bubbled as it collides with the wall surface of the sludge separation unit 14, and passes through an opening of the barrier plate 11.

The sludge and water passing through the barrier plate 11 are introduced towards the sludge processing unit 13 through the water tray 111. The sludge and water passing through the water tray 111 are discharged to an opposite side of the sludge guide 23, and are dropped to an outer surface of the drum 21.

The drum 21 may be rotated, and the drum 21 is rotated by driving the drum motor 211. The sludge dropped by rotating the drum 21 is attached to the outer surface of the drum 21, and the water is discharged through the vent holes of the drum 21 and is stored at a lower portion of the sludge processing unit 13. The sludge mixed with the water stored in the sludge processing unit 13 may be attached to the outer surface of the drum 21 while the drum 21 is rotated. The water stored at a lower portion of the sludge processing unit 13 may be continuously sterilized by an operation of the ultraviolet lamp 131.

The water stored in the sludge processing unit 13 may be supplied to the water supply nozzle 151 along the water supply pipe 15 by the water supply pump 152 and be mixed with the contaminated air compulsorily blown by the main blower 105, and then passes through the sludge separation unit 14 and is introduced into the sludge processing unit 13 to circulate.

Meanwhile, the sludge attached to the outer surface of the drum 21 may be discharged to the outside by the sludge discharge unit 20. In detail, the air is discharged from the air nozzle 22 by driving the sludge blower 221 while the sludge is attached to the outer surface of the drum 21. Then, the air discharged from the air nozzle 22 is ejected from the inner side of the drum 21 to the outside, and the air passes through the vent holes of the drum 21 and the sludge attached to the outer surface of the drum 21 is separated from the drum 21 and is sprayed.

The sludge separated from the drum 21 is moved to the sludge guide 23 by the air ejected from the air nozzle 22, and is collected in the screw feeding unit 24 by the sludge guide 23.

The screw member 242 is rotated by driving the screw motor 241, and the sludge collected in the screw case 243 is fed by rotating the screw member 242 and is moved to the inside of the sludge box 25 outside the case 10.

Accordingly, the sludge continuously generated in the interior of the case 10 is discharged to the sludge box 25 and is collected, and the user can evacuate or process the sludge collected in the sludge box 25 at once.

Hereinafter, the second embodiment of the present invention will be described.

The second embodiment of the present invention is the same as the first embodiment of the present invention except for the structure of the sludge processing unit, and thus the same structures are denoted by the same reference numerals and a description thereof will be omitted.

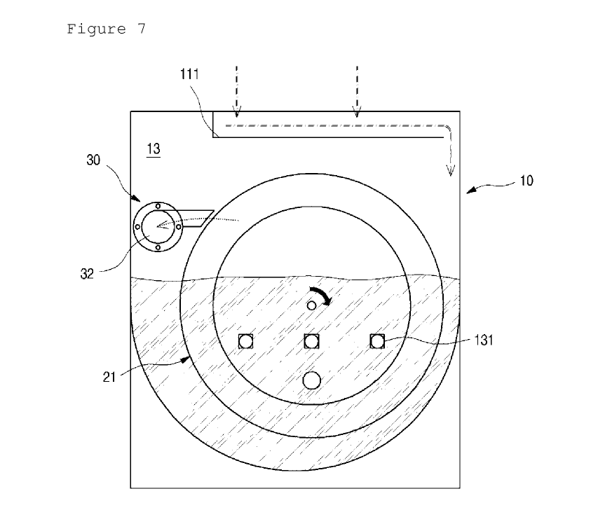

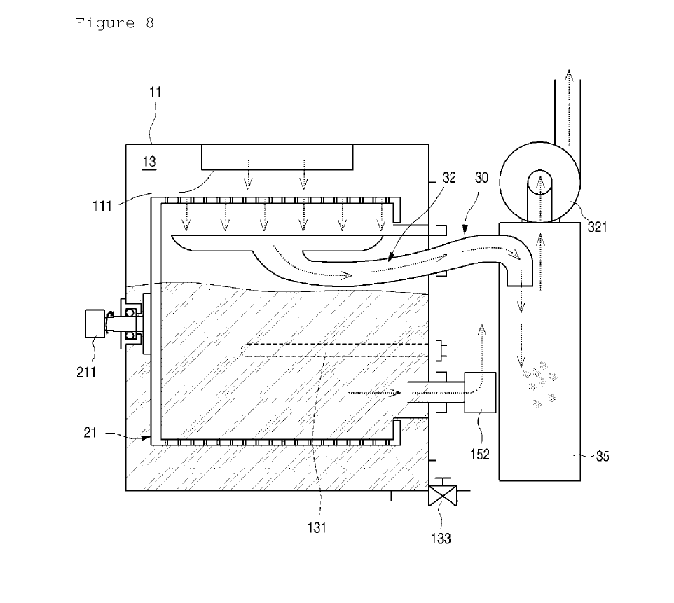

FIG. 7 is a side view illustrating a structure of a sludge processing unit according to a first embodiment of the present invention. FIG. 8 is a front view illustrating the structure of the sludge processing unit.

A drum 21 driven by a drum motor 211 is provided in the interior of a sludge processing unit 13 of the dust collecting apparatus 1 according to the second embodiment of the present invention. An ultraviolet lamp 131, a water supply pump 152, and a water discharge value 133 may be provided in the case 10.

Meanwhile, a sludge discharge unit 30 is provided in the sludge processing unit 13. The sludge discharge unit 30 may include a drum 21, a sludge box 35 in which sludge is collected, a suction blower 321 that generates suction force, and a suction nozzle 32 that suctions the sludge on the outer surface of the drum 21.

The drum 21 is eccentrically arranged inside the sludge processing unit 13, and is mounted to be rotated. A plurality of holes is formed on an outer surface of the drum 21 such that water is discharged through the holes and sludge is attached on the outer surface of the drum 21.

The sludge box 35 is provided outside the case 10, and provides a sufficient space such that the sludge is collected in the sludge box 35. The sludge box 35 is communicated with the suction blower 321 and the suction nozzle 32.

The suction blower 321 is communicated with an upper surface of the sludge box 35, and the suction nozzle 32 provides a suction force by which the sludge attached to the outside of the drum 21 may be suctioned. The suction nozzle 32 is located at an upper portion of the inside of the sludge processing unit 13, and is arranged adjacent to the outer surface of the drum 21. Then, the suction nozzle 32 may extend lengthily along a transverse direction of the drum 21, and an entrance of the suction nozzle 32 may be formed to correspond to a length in the transverse direction of the drum 21.

Meanwhile, an exit of the suction nozzle 32 is connected to the sludge box 35, and then the exit of the suction nozzle 32 is connected to an upper surface or an upper portion of the sludge box 35 such that the sludge introduced through the suction nozzle 32 may be dropped in the interior of the sludge box 35.

Accordingly, the contaminated air mixed with the water introduced into the case 10 collides with the sludge separation unit 14 inside the case 10 and is separated into air, water, and sludge, and the separated water and sludge are introduced into the sludge processing unit 13 through the water tray 111.

The sludge is attached to a surface of the drum 21 by rotating the drum 21, and the sludge attached to the surface of the drum 21 is suctioned into the suction nozzle 32 by driving the suction blower 321. The sludge suctioned into the suction nozzle 32 is introduced into the sludge box 35 along the suction nozzle 32, and the sludge introduced into the sludge box 35 is dropped by the weight thereof and is collected in the interior of the sludge box 35 and the air separated from the sludge is discharged to the outside through the suction blower 321.

Then, the discharge duct 322 of the suction blower 321 may be connected to one side of the pipe of the main blower 105 or the connection duct 106, and accordingly, the air discharged through the suction blower 321 may be purified in the inside of the case 10 again.

Hereinafter, the third embodiment of the present invention will be described.

The third embodiment of the present invention is the same as the first embodiment of the present invention except for the structure of the sludge processing unit, and thus the same structures are denoted by the same reference numerals and a description thereof will be omitted.

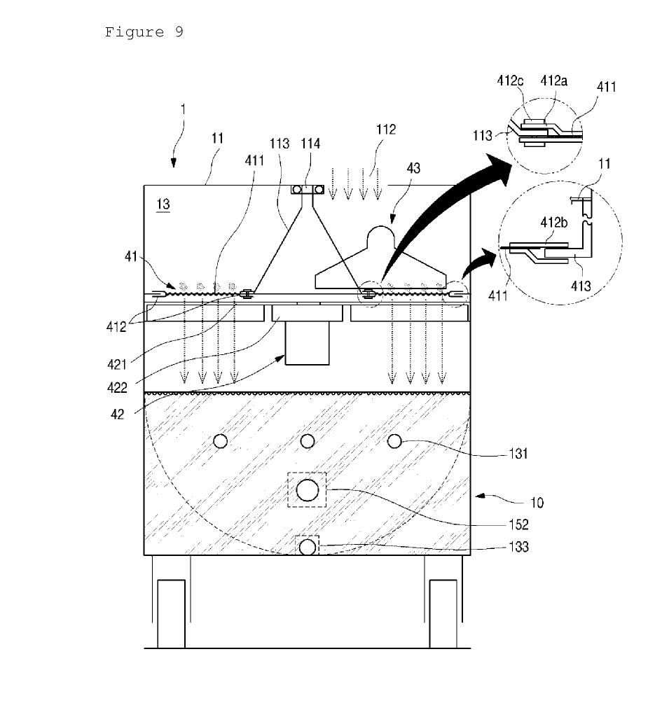

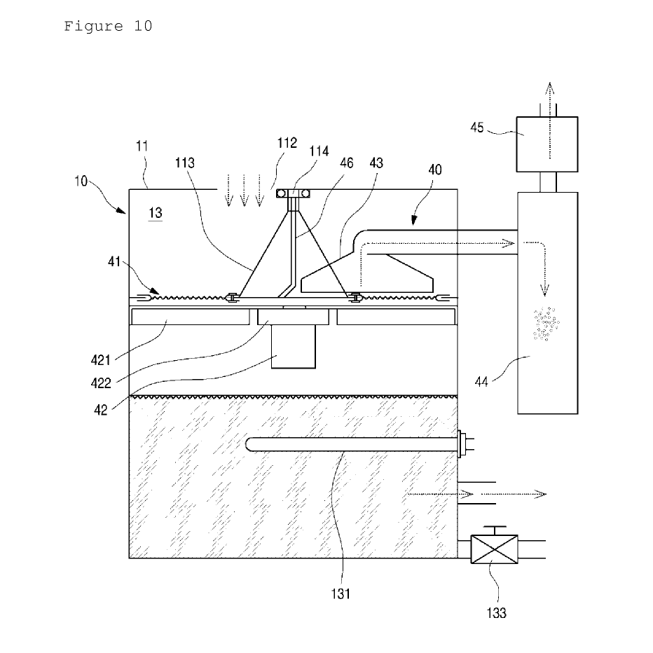

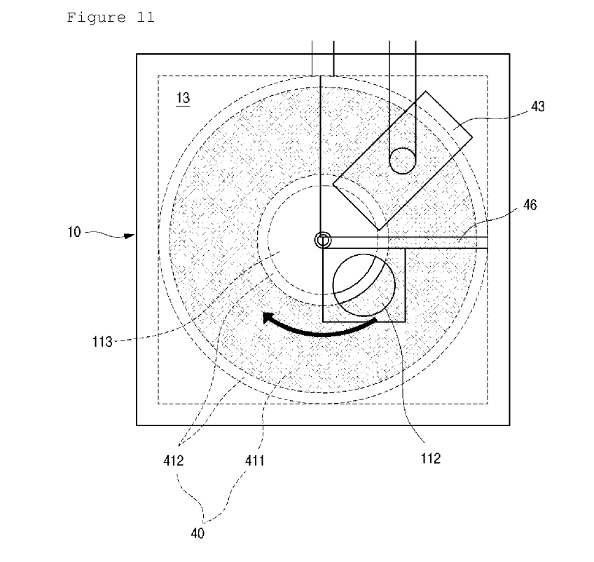

FIG. 9 is a side view illustrating a structure of a sludge processing unit according to a third embodiment of the present invention. FIG. 10 is a side view of the sludge processing unit. FIG. 11 is a plan view of the sludge processing unit.

As illustrated in the drawings, a case 10 of the dust collecting apparatus 1 according to the third embodiment of the present invention may include an ultraviolet lamp 131, a water supply pump 152, and a discharge value 133.

A barrier plate 11 that partitions the interior of the case 10 into an air flow unit 12 and a sludge processing unit 13 may be provided in the interior of the case 10. An inlet 112 through which sludge and water are dropped downwards are further formed in the barrier plate 11. The inlet 112 may be provided at one of parts obtained by dividing the case 10 into four parts with reference to the center of the case 10, and may be opened to make contact with the center of the case 10.

The water guide 113 may be provided below the barrier plate 11. The water guide 113 is adapted to guide flows of the water and the sludge introduced through the inlet 112, and may have a conic shape, the diameter of which becomes larger as it goes downwards. Accordingly, the water and the sludge introduced through the inlet 112 flow downwards along the water guide 113.

A bearing 114 is provided at an upper end of the water guide 113, and the water guide 113 may be connected to the barrier plate 11 to be rotated, by the bearing 114. Of course, the water guide 113 may be rotatably connected to the barrier plate 11 by a configuration other than the bearing 114. A lower end of the water guide 113 is coupled to a screen motor 42, which will be described below, and may be rotated by the screen motor 42.

Meanwhile, a sludge processing unit 40 is provided in the sludge discharge unit 13. The sludge discharging unit 40 may include a screen 41 that separates dropped sludge and water and filters the sludge, a screen motor 42 that rotates the screen 41, a suction member 43 for suctioning the sludge on the screen, and a sludge box 44 and a suction blower 45 connected to the suction member 43.

In detail, the screen has a disk shape, and may have a mesh or net shape having a plurality of vent holes. Accordingly, the water passes through the screen 41, and the sludge is left on the upper surface of the screen 41. The screen 41 may have a disk shape having a diameter corresponding to the horizontal distance of the case 10, and is fixedly coupled to a lower end of the water guide 113 and is mounted to be rotatable together with the water guide 113 when the screen motor 42 provided below the screen 41 is rotated.

The screen motor 42 is fixed to the case 10 by a motor frame 421, and may be arranged at a central portion of the case 10. A rotary shaft of the screen motor 42 may be connected to a central portion of the screen 41 or a central portion of the water guide 113, and a reducer 422 may be further provided between the screen motor 42 and the screen to adjust the rotational speed of the screen 41.

Meanwhile, the screen 41 may include a plate 411 having a plurality of vent holes and having an opened central portion, and a screen frame 412 formed to cross the opened central portion of the screen 41 and to be coupled to the screen motor 42, and further formed along the inner and outer circumferences of the plate 411.

The screen frame 412 may include an inner frame 412a formed along an inner surface of the screen 41, and an outer frame 412b formed along an outer surface of the screen 41.

The inner frame 412a may accommodate an end of the water guide 113 bent from a lower end thereof to the outside, and may be coupled to the inner frame 412a by a coupling member 412a passing through the water guide 113. Of course, if necessary, the inner frame 412a and a lower end of the water guide 113 may be coupled to each other through welding or bonding.

The outer frame 412b accommodates a bent end of a screen bracket 413 fixed to the case 10. Then, the outer frame 412b and the end of the screen bracket 413 may not be fixed to each other but may be mounted on each other to be relatively moved when the screen 41 is rotated. If necessary, coating or an additional pad that may lower frictional coefficient to make the rotation of the screen 41 smooth may be further provided on the inner side of the outer frame 412b.

Accordingly, the screen 41 is coupled and fixed to the water guide 113 by the screen frame 412, and may be rotatably supported by an end of the screen bracket 413. Because the outer frame 412b and an end of the screen bracket 413 are not fixed, they may be relatively moved and may allow for rotation of the screen 41 and the water guide 113.

The screen bracket 413 is fixedly mounted on a lower surface of the barrier plate 11 of the case 10. The screen bracket 413 may extend downwards to a location of the screen 41 and the extending end of the screen bracket 413 may be bent towards the screen 41. The screen bracket 413 may not be formed in some sections in the interior of the case 10 so as not to interfere with the suction member 43 in the interior of the case 10.

A blocking plate 46 may be further formed at one side of an upper portion of the screen 41. The blocking plate 46 extends downwards from a lower end of the inlet 112 to the screen 41. The water guide 113 and the screen bracket 413 are connected to each other so that the sludge and water introduced through the inlet 112 can be prevented from being directly introduced to the suction member 43.

Meanwhile, the suction member 43 is provided above the screen 41, and is communicated with the sludge box 44. One end of the suction member 43 is arranged at a location adjacent to the upper surface of the screen 41, and an entrance of the suction member 43 is wide such that the sludge on the upper surface of the screen may be easily suctioned.

The sludge box 44 is provided outside the case 10, and provides a sufficient space such that the sludge is collected in the sludge box 35. The sludge box 44 may be connected to the suction blower 45 and the suction member 43.

Accordingly, the contaminated air mixed with the water introduced into the case 10 collides with the sludge separation unit 14 inside the case 10 and is separated into air, water, and sludge, and the separated water and sludge are dropped through the inlet 112 and introduced into the sludge processing unit 13.

Then, the water and the sludge are moved along the water guide 113 to reach the screen 41, and are prevented from being directly introduced into the suction member 43 by the blocking plate 46. Accordingly, among the water and the sludge that reached the screen 41, the water passes through the screen 41 to be collected at a lower portion of the sludge processing unit 13 and the sludge is left in the screen 41.

The sludge left in the screen 41 may move to the suction member 43 according to rotation of the screen 41. If the sludge on the screen 41 reaches a location of the suction member 43, it is suctioned by the suction member 43 by driving the suction blower 45. The sludge suction into the suction member 43 is introduced into the sludge box 44 along the suction member 43, the sludge introduced into the sludge box 44 is dropped by the weight thereof and is collected in the interior of the sludge box 44, and the air separated from the sludge is discharged to the outside through the suction blower 45 and the discharged air may be communicated with the connection duct 106 of the above-mentioned embodiment.

Hereinafter, the fourth embodiment of the present invention will be described.

The fourth embodiment of the present invention is the same as the first embodiment of the present invention except for the structure of the air flow unit, and thus the same structures are denoted by the same reference numerals and a description thereof will be omitted.

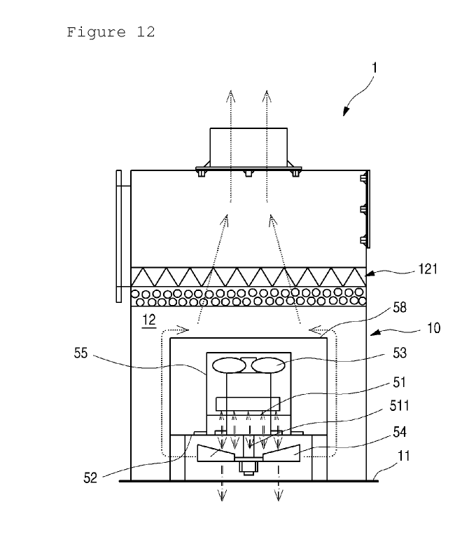

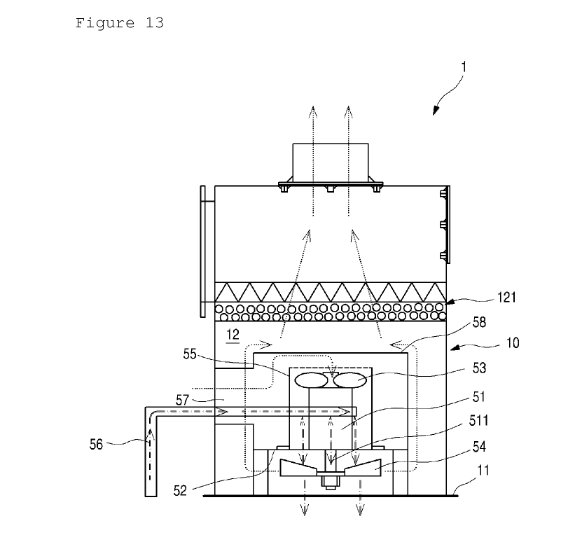

FIG. 12 is a front view illustrating a structure of an air flow unit according to a fourth embodiment of the present invention. FIG. 13 is a side view of the air flow unit.

As illustrated in the drawings, in the dust collecting apparatus 1 according to the fourth embodiment of the present invention, an upper space of the case 10 is defined as the air flow unit 12 by the barrier plate 11 that partitions the interior of the case 10.

A filter assembly 121 is provided at an upper portion of the air flow unit 12, and an internal motor 51 is provided below the filter assembly 121. A motor having a waterproof structure may be used as the internal motor 51 so that the internal motor 51 is not influenced by the water supplied from the outside. The internal motor 51 is adapted to suction and compulsorily feed the contaminated air from the outside, and is mounted on a motor mount 52 of the barrier plate 11. A rotary shaft 511 of the internal motor 51 mounted on the motor mount 52 extends vertically, and a suction fan 53 for suctioning air is mounted on an upper end of the rotary shaft 511 and an impeller 54 is provided at a lower end of the rotary shaft 511.

The diameter of the impeller 54 is larger than that of the suction fan so that the contaminated air mixed with the water flowing downwards may pass while colliding with the blades of the impeller 54. Accordingly, the impeller 54 compulsorily feeds the suctioned air and collides with the contaminated air mixed with water to spray the contaminated air so that the sludge in the contaminated air, the water, and the air may be separated.

A cylindrical motor cover 55 that accommodates the internal motor 51 and the suction fan 53 is provided on the upper surface of the motor mount 52. At least a portion of the upper surface of the motor cover 55 is opened so that the contaminated air is introduced through the upper surface of the motor cover 55. A water supply pipe that passes and extends through the motor cover 55 to supply the water mixed with the contaminated air extends in the interior of the motor cover 55.

The water supply pipe 56 is formed along the circumference of the internal motor 51 below the suction fan 53 to discharge the water downwards so that the contaminated air introduced through the suction fan 53 and the water flows downwards while being mixed.

An upper case 58 that accommodates the motor cover 55, including the motor mount 52, may be further formed, and the upper case 58 may be communicated with an introduction duct 57 through which the contaminated air passing through the case is introduced. Then, the upper case 58 may have a structure in which an upper portion thereof is sealed. Accordingly, the contaminated air is introduced into the upper case 58 through the introduction duct 57, and then may be introduced through the upper surface of the motor cover 55.

Meanwhile, a lower end of the upper case 58 is fixed to the barrier plate 11. At least a portion of a side of the impeller 54 is opened so that the air separated through the impeller 54 may pass through the opening.

Accordingly, the suction fan 53 is rotated by driving the internal motor 51 so that the contaminated air may be introduced through the introduction duct 57. Then, the introduced contaminated air passes through the upper case 58 and the motor cover 55 to be moved downwards, and is mixed with the water supplied through the water supply pipe 56 in the process.

The contaminated air mixed with the water is compulsorily fed downwards, and collides with the impeller 54 and sprayed in the process. Accordingly, the air, the sludge, and the water may be separated by the impeller 54, and the separated air flows to a side of the impeller 54 and passes through a lower end of the upper case 58. In this way, the air flowing to the outside of the upper case 58 passes through the filter assembly 121 and is discharged to the outside.

Meanwhile, the sludge and the water separated by the impeller 54 is dropped downwards by the weight thereof, and are introduced into the sludge processing unit 13. The sludge is discharged to the outside of the case 10 in the sludge processing unit 13.

Hereinafter, the fifth embodiment of the present invention will be described.

The fifth embodiment of the present invention is the same as the first embodiment of the present invention except for the structure of the air flow unit, and thus the same structures are denoted by the same reference numerals and a description thereof will be omitted.

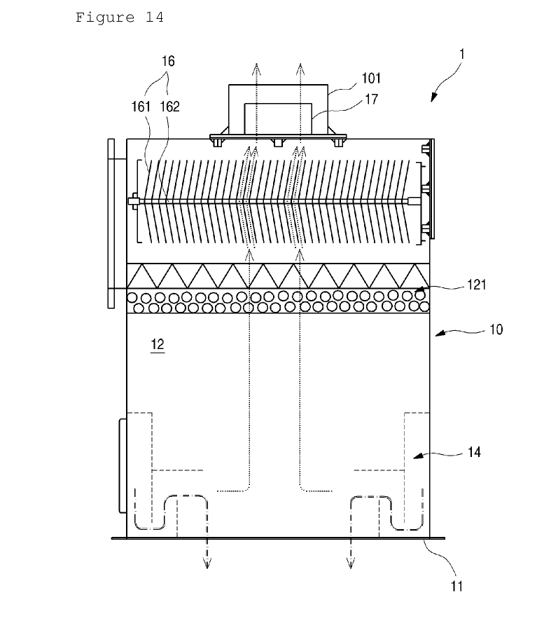

FIG. 14 is a front view illustrating a structure of an air flow unit according to a fifth embodiment of the present invention.

As illustrated in the drawings, in the dust collecting apparatus 1 according to the fourth embodiment of the present invention, an upper space of the case 10 is defined as the air flow unit 12 by the barrier plate 11 that partitions the interior of the case 10.

The sludge separation unit 14 is provided at a lower portion of the air flow unit 12, and the filter assembly 121 is provided above the sludge separation unit 14.

A photocatalytic module 16 is mounted into a space above the filter assembly 121. The photocatalytic module 16 may include a plurality of photocatalytic plates 161 continuously arranged by a predetermined interval, and an ultraviolet lamp 162 that passes through the plurality of photocatalytic plates 161.

The plurality of photocatalytic plates 161 is formed of a plate-shaped material coated with titanium dioxide, and the air passing through the filter assembly 121 passes through the photocatalytic plates 161 before passing through the discharge hole 101 on the upper surface of the case 10. Then, the plurality of photocatalytic plates 161 may be arranged to have a suitable number a predetermined interval to increase a contact area with the passing air.

Meanwhile, opposite sides of the photocatalytic plates 161 are bent in the same direction with respect to the center thereof, and the bending angle may be about 5 to 15 degrees. Accordingly, when the air flows along a passage formed by the plurality of photocatalytic plates 161, it may smoothly contact the photocatalytic plates 161.

A plurality of air holes may be punched along the entire photocatalytic plates 161 so that air may pass through the air holes. The air flowing through the passage formed by the adjacent photocatalytic plates 161 may flow other adjacent passages through the air holes, and in the process, an entire contact area may increase through a contact with the photocatalytic plates 161 to make the photocatalytic reaction more active.

If the ultraviolet lamp 162 passes through the plurality of photocatalytic plates 161 to be turned on, an active photocatalytic reaction may occur in the photocatalytic plates 161. Accordingly, the air passing through the photocatalytic plates 161 may be deodorized and sterilized, and may be introduced through the discharge hole 101 while being fresh.

Meanwhile, a negative ion generator 17 may be further provided on one side of the discharge hole 101, and the air passing through the discharge hole 101 may be neutralized again by an operation of the negative ion generator 17 so that the further purified air may be discharged through the discharge hole 101.

Hereinafter, the fifth embodiment of the present invention will be described.

The fifth embodiment of the present invention is the same as the first embodiment of the present invention except for the structure of the photocatalytic purifying unit, and thus the same structures are denoted by the same reference numerals and a description thereof will be omitted.

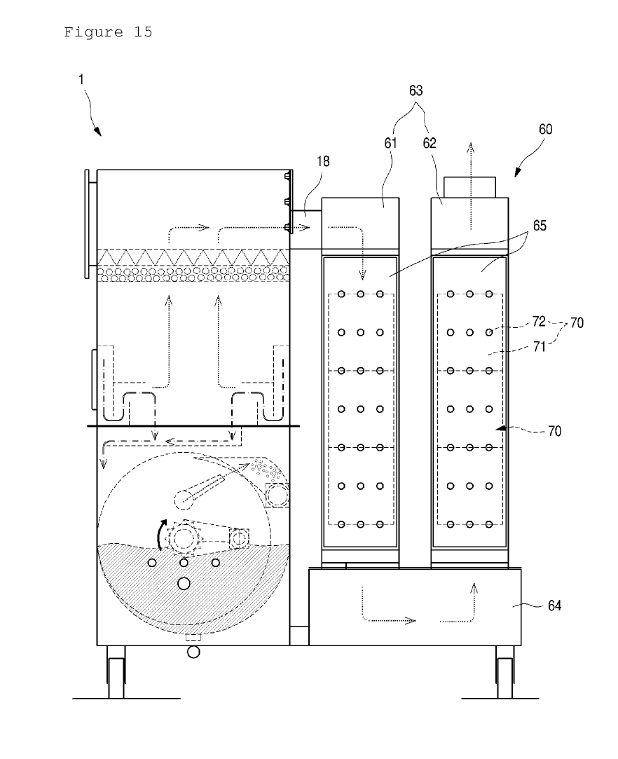

FIG. 15 is a front view of an air purifying apparatus according to a sixth embodiment of the present invention.

As illustrated in the drawings, the dust collecting apparatus 1 according to the sixth embodiment of the present invention may further include a photocatalytic purifying unit 60. Then, the configuration of the dust collecting apparatus 1 is the same as the dust collecting apparatus 1 of the above-mentioned embodiments, and thus a detailed description thereof will be omitted.

A discharge pipe 18 is formed in the case 10 of the dust collecting apparatus 1, and the discharge pipe 18 has a structure connected to the photocatalytic purifying unit 60. The discharge pipe 18 connects the dust collecting apparatus 1 and the photocatalytic purifying unit 60, and accordingly, the air from which the sludge and the contaminants are separated in the dust collecting apparatus 1 may be supplied to an upper portion of the photocatalytic purifying unit 60.

The photocatalytic purifying unit 60 may include a base 64 that forms the bottom thereof, and a purifying unit 63 that is provided on the upper surface of the base 64 to form a flow passage of air and in which the photocatalytic module 70 is accommodated. The number of the purifying units 63 may be one or more according to the required air purifying performance, and it will be exemplified in the embodiment of the present invention for convenience of description and understanding that the purifying unit 63 includes a first purifying unit 61 and a second purifying unit 62.

The base 64 may be connected to the case of the dust collecting apparatus 1, and the same caster 134 as the caster provided on the bottom of the case 10 may be provided on the bottom of the base 64 to facilitate movement of the dust collecting apparatus 1.

The base 64 forms a predetermined space therein, and the air passing through the first purifying unit 61 may be introduced into the second purifying unit 62 via the base 64. To achieve this, an opening communicated with the first purifying unit 61 and the second purifying unit 62 may be formed on the upper surface of the base 64.

The purifying unit 63 has a structure in which the plurality of photocatalytic modules 70 are stacked, and the plurality of photocatalytic modules 70 may be attached through a door 65 that may open or close one surface of the purifying unit 63. That is, the number of stacked photocatalytic modules 70 in the purifying unit 63 maybe adjusted by opening the door 65. The number of the ultraviolet lamps 72 constituting the photocatalytic module 70 may be conveniently adjusted by opening and closing the door 65.

The first purifying unit 61 is connected to the case 10 to form a passage of air flowing downwards, and then the flowing air passes through all the plurality of photocatalytic modules 70 that are stacked and is guided to be introduced into the base 64.

The second purifying unit 62 is connected to the case 10 to form a passage of air flowing upwards, and then the flowing air passes through all the plurality of photocatalytic modules 70 that are stacked and is guided to be introduced into the base 64.

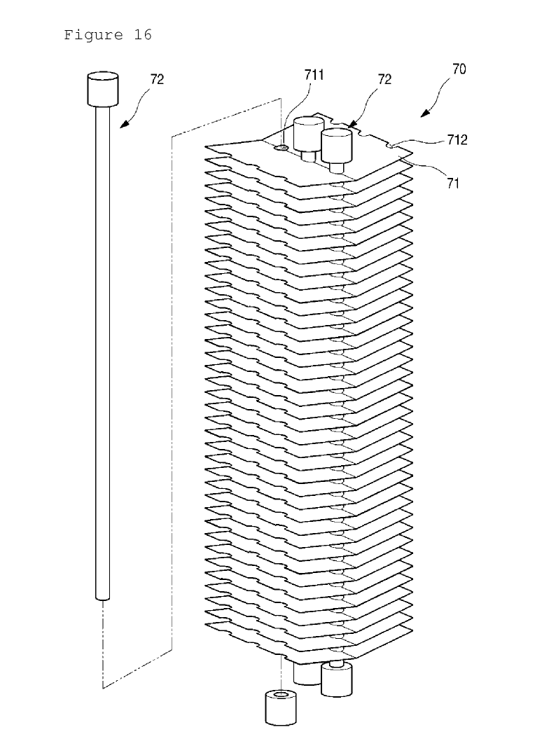

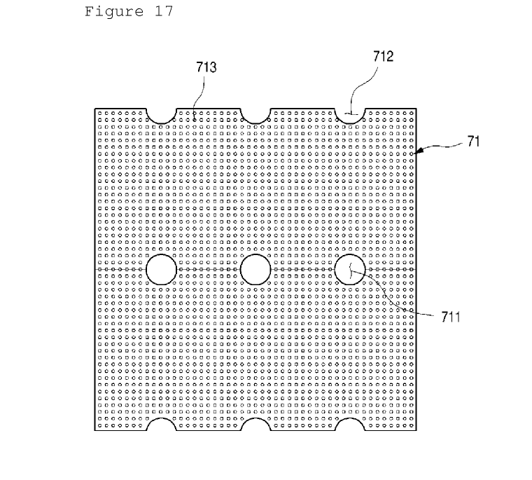

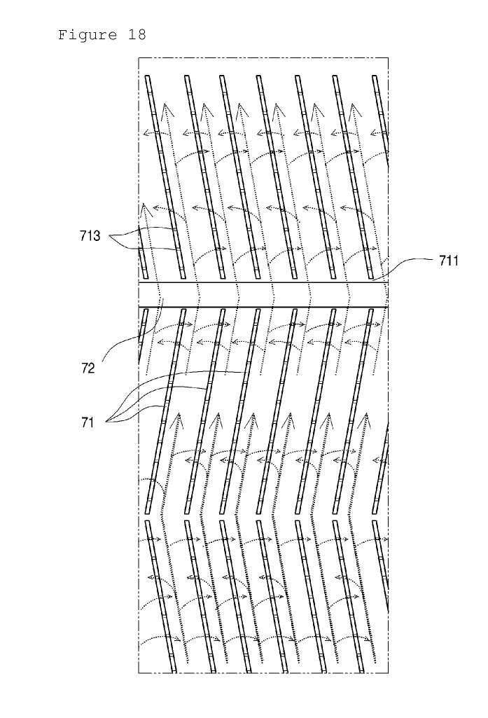

FIG. 16 is an exploded perspective view of a photocatalytic module according to the sixth embodiment of the present invention. FIG. 17 is a plan view of a photocatalytic plate that is a main part of the photocatalytic module. FIG. 18 is a partially sectional view of the photocatalytic module.

Referring to the drawings, in a configuration of the photocatalytic module 70, the photocatalytic module 70 may include a plurality of photocatalytic plates 71 continuously arranged at a predetermined interval, and an ultraviolet lamp 72 that passes through the plurality of photocatalytic plates 71.

The photocatalytic plates 71 are adapted to make the photocatalytic reaction active when the ultraviolet lamp 71 is turned on, and opposite surfaces thereof may be coated with titanium dioxide, which may cause a photocatalytic reaction. The photocatalytic plates 71 may have a width corresponding to the width of the purifying unit 63 to occupy the interior of the purifying unit 63.

Meanwhile, the photocatalytic plates 71 have a substantially thin plate shape. The plurality of photocatalytic plates 71 constituting the photocatalytic module 70 has the same shape. Opposite sides of the photocatalytic plates 71 are bent in the same direction with respect to the center thereof. That is, the opposite sides of the photocatalytic plates 71 are inclined with respect to the center thereof.

Then, the bending angle of the photocatalytic plates 71 is about 5 to 15 degrees. Accordingly, when the air flows along a passage formed by the plurality of photocatalytic plates 71, it may smoothly contact the photocatalytic plates 71.

A plurality of air holes 713 may be punched along the entire photocatalytic plates 71 so that air may pass through the air holes. As illustrated in FIG. 18, the air flowing through the passage formed by the adjacent photocatalytic plates 71 may flow other adjacent passages through the air holes 713, and in the process, an entire contact area may increase through a contact with the photocatalytic plates 71 to make the photocatalytic reaction more active.

A lamp mounting hole 711 through which the ultraviolet lamp 71 may pass to be mounted are formed at the centers of the photocatalytic plates 71. One or more lamp mounting holes 711 may be formed, and three lamp mounting holes 711 may be formed along the bent centers of the photocatalytic plates 71 as illustrated in the drawings.

Lamp mounting recesses 712 for forming holes in which the ultraviolet lamps 72 may be mounted to contact each other when the photocatalytic modules 70 are stacked are formed at opposite ends of the photocatalytic plates 71. One or more lamp mounting recesses 712 may be formed at ends of the photocatalytic plates 71, and three lamp mounting recesses 712 may be formed at opposite ends of the photocatalytic plates 71 as illustrated in the drawings.

Meanwhile, the ultraviolet lamp 72 is mounted to pass through the plurality of photocatalytic plates 71. The ultraviolet lamp 72 may be mounted on the lamp mounting holes 711 and/or the lamp mounting recesses 712, and one ultraviolet lamp 72 is mounted to pass through all the photocatalytic plates 71. Then, the number of the ultraviolet lamps 72 mounted on the photocatalytic module 70 may be determined through selection of the user, and the ultraviolet lamp 72 may be mounted to a desired location of the plurality of lamp mounting holes 711 and the lamp mounting recesses 712.

That is, the number of the ultraviolet lamps 72 mounted to the lamp mounting holes 711 and the lamp mounting recesses 712 is increased to increase the purifying performance of the photocatalytic purifying unit 60, and the number of the ultraviolet lamps 72 mounted to the lamp mounting holes 711 and the lamp mounting recesses 712 is decreased to decrease the purifying performance of the photocatalytic purifying unit 60.

In this way, the mounting and separation of the ultraviolet lamps 72 may be determined and manipulated by the user, and a door 65 is opened in the purifying unit 63 to facilitate the mounting and separation of the ultraviolet lamps 72, and an end of the photocatalytic module 70 is exposed to easily mount and separate the photocatalytic module 70 when the door 65 is opened.

Meanwhile, the photocatalytic plates 71 between the adjacent photocatalytic modules 70 may contact each other when the photocatalytic modules 70 are stacked such that the flow path of the air may be continuous. A hole in which the ultraviolet lamp 72 may be mounted may be formed.

The photocatalytic module 70 may be mounted and separated by opening the door 65, and the photocatalytic module 70 may be inserted into and separated from the purifying unit 63 while the door is opened.

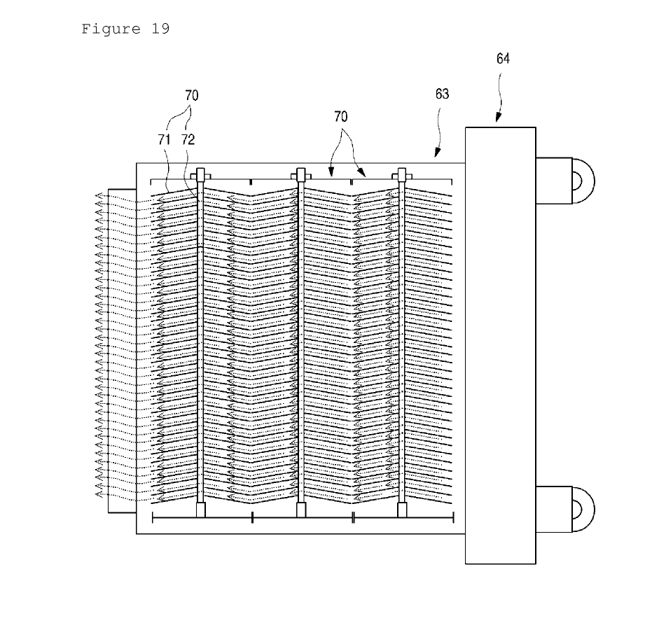

FIG. 19 is a view illustrating an air flow in the photocatalytic module.

As illustrated in the drawings, the air that passes through the case 10 of the dust collecting apparatus 1 such that foreign substances are removed from the air flows along the purifying unit 63, and is sterilized while passing through the photocatalytic module 70 in the interior of the purifying unit 63.

In detail, the air flowing along the purifying unit 63 from one end of the purifying unit 63 passes through the photocatalytic modules 70 that are continuously stacked. Then, the ends of the photocatalytic plates 71 of the adjacent photocatalytic modules 70 contact each other to form a passage.

The air flowing along the purifying unit 63 contacts all the photocatalytic plates 71. In particular, a central portion of the photocatalytic plate 71 is bent, and the flow direction of the flowing air is continuously changed in zigzag, and then the air collides with the photocatalytic plate 71.

Accordingly, the photocatalytic reaction of the photocatalytic plate 71 by an operation of the ultraviolet lamp 72 effectively sterilizes the air flowing along the purifying unit 63. The air sterilized in this way may be discharged via the purifying unit 63.

Meanwhile, when a plurality of purifying units 63 are provided, the air continuously flows along the purifying unit 63, and accordingly, a further improved purifying performance can be implemented. The user may set the number of the purifying units 63 in consideration of the characteristics, and the numbers of the photocatalytic modules 70 and the violet lamps 72 accommodated in the purifying units 63 may be adjusted.

Furthermore, if necessary, the photocatalytic purifying unit 60 may be separately configured from the dust collecting apparatus 1 to be operated alone, and then a separate passage for introduction of the air and a blower may be installed at one side of the purifying unit 63.

### Industrial Applicability

According to an embodiment of the present invention, air purifying performance can be improved and convenience of use can be enhanced, and accordingly, the present invention can be industrially applicable.

1. An air purifying apparatus comprising:
a case that forms an external appearance of the air purifying apparatus and has a discharge hole through which purified air is discharged;
a main blower that is communicated with the interior of the case such that contaminated air flows into the case;
a water supply pipe that is provided at an outlet of the main blower to supply water mixed with the contaminated air introduced into the case;
a sludge separation unit that is provided in an interior space of the case to form a passage in which the contaminated air mixed with the water introduced into the case makes continuous collisions and to separate the water in the contaminated air and the sludge;
a barrier plate that partitions the interior space of the case into an upper air introduction unit and a lower sludge processing unit below the sludge separation unit and has a passage through which the water and the sludge separated from the contaminated air passes; and
a sludge discharge unit that is provided in the sludge processing unit to continuously separate the water and the sludge passing through the base plate by rotation thereof and to discharge the separated sludge to the outside of the case,
wherein the sludge discharge unit comprises:
a drum that is rotated in the sludge processing unit and has a plurality of holes on an outer surface thereof;
an air nozzle that is provided inside the drum to eject air supplied by a sludge blower provided in the case and to spray the sludge attached to the outer surface of the drum;
a sludge guide that is provided outside the drum facing the air nozzle to guide the sprayed sludge;
a screw feeding unit that is provided below the sludge guide to horizontally move the sludge collected by the sludge guide in a screwed manner; and
a sludge box that is connected to the screw feeding unit to store the fed sludge.

2. (canceled)

3. The air purifying apparatus of claim 1, wherein the sludge separation unit includes a plurality of plate-shaped structures that is arranged in a direction perpendicular to a flow direction of air introduced from an outlet of the main blower.

4. The air purifying apparatus of claim 3, wherein the sludge separation unit is formed along a circumference of the case, and a plurality of holes, through which water and sludge are dropped, are formed in an inner area of the sludge separation unit in the barrier plate.

5. The air purifying apparatus of claim 1, wherein a water tray that guides the water and the sludge moved downwards to one side of the sludge discharge unit is provided on a lower surface of the barrier plate.

6-7. (canceled)

8. The air purifying apparatus of claim 1, wherein all of the air nozzle, the sludge guide, and the screw feeding unit extend in an axial direction of the drum.

9. The air purifying apparatus of claim 1, wherein the screw feeding unit comprises:
a screw member that is directly connected to a screw motor provided in the case and has a spiral shape to feed the sludge while being rotated; and
a screw case that accommodates at least a portion of the screw member below the screw member.

10-18. (canceled)

19. The air purifying apparatus of claim 1, wherein a photocatalytic module for purifying the discharged air is provided at an upper portion of the air flow unit, and the photocatalytic module comprises a plurality of photocatalytic plates coated with titanium dioxide and at least one ultraviolet lamps passing through the plurality of photocatalytic plates.

20. (canceled)

21. The air purifying apparatus of claim 1, further comprising:
a photocatalytic purifying unit for purifying the air from which water and sludge are separated on an outer side of the case,
wherein the photocatalytic purifying unit comprises a purifying unit in which a plurality of photocatalytic modules each comprising a plurality of photocatalytic plates coated with titanium dioxide and at least one ultraviolet lamps passing through the plurality of photocatalytic plates are vertically arranged in parallel to each other.

22. The air purifying apparatus of claim 21, wherein a plurality of purifying unit are arranged in parallel in the photocatalytic purifying unit, and a base that is communicated with the plurality of purifying units to form flow passages of the air between the plurality of purifying units is further provided.

23. The air purifying apparatus of claim 21, wherein a door that is provided to be opened and closed such that an outer end of the ultraviolet lamp is exposed is provided in the purifying unit.

24. The air purifying apparatus of claim 23, wherein the photocatalytic module is arranged such that a surface of the photocatalytic plate, into which the ultraviolet lamp is inserted, is exposed when the door is opened.

25. The air purifying apparatus of claim 21, wherein the plurality of photocatalytic modules are vertically stacked such that ends of the vertically adjacent photocatalytic plates contact each other to connect the flow passages of the air.

26. (canceled)

27. The air purifying apparatus of claim 25, wherein a plurality of air holes are punched over the front surface of the photocatalytic plate, and the photocatalytic plate is bent along a central portion thereof and opposite side surfaces thereof are inclined with respect to the central portion thereof.

28. The air purifying apparatus of claim 25, wherein one or more lamp mounting recesses that are recessed such that a hole into which the ultraviolet lamp is inserted is formed when the plurality of photocatalytic modules are stacked are formed at adjacent ends of the plurality of photocatalytic modules.

29-33. (canceled)

34. The air purifying apparatus of claim 25, wherein at least one lamp mounting hole that is opened along a central portion of the photocatalytic plate and into which the ultraviolet lamp is inserted is formed in the photocatalytic plate.
